(19) United States
(12) Patent Application Publication
(10) Pub. No.: US 2026/0256773 A1
(43) Pub. Date: Sep. 3, 2026

(54) COMPOSITIONS COMPRISING LYSERGIC ACID DIETHYLAMIDE, OPTIONALLY IN COMBINATION WITH N-ACYLETHANOLAMINES AND USES THEREOF

(71) Applicant: Clearmind Labs Corp., Vancouver (CA)

(72) Inventor: Adi ZULOFF-SHANI, Tel Aviv (IL)

(21) Appl. No.: 19/154,426

(22) PCT Filed: Feb. 9, 2024
(86) PCT No.: PCT/IB2024/051238

§ 371 Date: Aug. 7, 2025

Related U.S. Application Data
(60) Provisional application No. 63/484,389, filed on Feb. 10, 2023.

(51) Int. Cl. A61K 31/48; A61K 31/16; A61K 31/164

(52) U.S. Cl. CPC A61K 31/48; A61K 31/16; A61K 31/164

(57) ABSTRACT

The present disclosure relates to pharmaceutical compositions comprising lysergic acid diethylamide (LSD), or a salt thereof, and combinations of LSD, or a pharmaceutically acceptable salt thereof and an N-acylethanolamine, or a salt thereof, and at least one pharmaceutically acceptable carrier and/or excipient, and their use in methods of treating alcoholism, depression, anxiety, headaches, and pain by administrating said compositions to a subject in need thereof in a therapeutically effective amount.

### CROSS-REFERENCE TO RELATED APPLICATIONS

This application claims the benefit of priority of U.S. Provisional Patent Application No. 63/484,389, filed on Feb. 10, 2023, the contents of which are incorporated herein in their entirety.

### FIELD OF THE DISCLOSURE

The present disclosure relates to compositions and methods for potentiating therapeutic effects and/or reducing side-effects of lysergic acid diethylamide (LSD) or a pharmaceutically acceptable salt thereof. The present disclosure provides pharmaceutical compositions comprising LSD, or pharmaceutically acceptable salts thereof, or combinations of LSD, or a pharmaceutically acceptable salt thereof and N-acylethanolamines, or pharmaceutically acceptable salts thereof, for example, palmitoylethanolamide (“PEA”) and methods for their use in a variety of indications amenable to treatment, including, but not limited to depression, anxiety, headaches, alcoholism, and pain.

### BACKGROUND OF THE DISCLOSURE

Psychoactive drugs such as Lysergic acid diethylamide (LSD) exert their effects primarily through agonist activity at serotonin 2A (5HT2A) receptors. The chemical structure of LSD is:

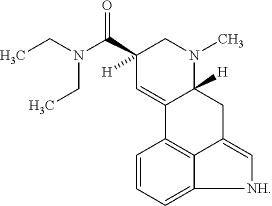 [SMILES: CCN(CC)C(=O)[C@@H]1C=C2c3cccc4[nH]cc(c34)C[C@H]2N(C)C1]

Controlled clinical trials have shown that LSD could produce a consistent and clinically significant beneficial effect in managing addiction. In particular, LSD could have a potentially beneficial effect on alcohol misuse and abuse. (Krebs, Teri S, and Pål-Ørjan Johansen. Lysergic acid diethylamide (LSD) for alcoholism: meta-analysis of randomized controlled trials. Journal of psychopharmacology (Oxford, England) vol. 26, 7 (2012): 994-1002).

At the same time, studies have suggested that LSD could have potential beneficial effects on mood and anxiety disorders, such as treatment-resistant depression or anxiety associated with life-threatening diseases. (Santos et al., Antidepressive, anxiolytic, and antiaddictive effects of ayahuasca, psilocybin and lysergic acid diethylamide (LSD): a systematic review of clinical trials published in the last 25 years. Ther Adv Psychopharmacol. 2016 June; 6(3):193-213). In addition, LSD have been associated with the termination period of cluster headaches or their extension of remission periods. (Sewell et al., Response of cluster headache to psilocybin and LSD. Neurology vol. 66, 12 (2006): 1920-2). Finally, it has been suggested that LSD may play a role in the management of chronic or persistent pain via interaction with nociceptive or antinociceptive processing. (Whelan et al., Lysergic acid diethylamide and psilocybin for the management of patients with persistent pain: a potential role? Pain management vol. 8, 3 (2018): 217-229).

N-acylethanolamines (NAEs) are lipid-derived signaling molecules. They are formed when one of several types of acyl groups is linked to the nitrogen atom of ethanolamine. Examples of N-acylethanolamines include anandamide (the amide of arachidonic acid (20:4 omega-6) and ethanolamine), N-Palmitoylethanolamine (the amide of palmitic acid (16:0) and ethanolamine), N-Oleoylethanolamine (the amide of oleic acid (18:1) and ethanolamine), N-Stearoylethanolamine (the amide of stearic acid (18:0) and ethanolamine) and N-Docosahexaenoylethanolamine (the amide of docosahexaenoic acid (22:6) and ethanolamine).

Palmitoylethanolamide (PEA, also known as N-(2-hydroxyethyl) hexadecanamide; Hydroxyethylpalmitamide; palmidrol; N-palmitoylethanolamine; and palmitoylethanolamide) is an endogenous fatty acid amide, belonging to the class of nuclear factor agonists. The chemical structure of PEA is:

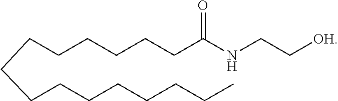 [SMILES: CCCCCCCCCCCCCCCC(=O)NCCO]

PEA has been demonstrated to bind to a receptor in the cell nucleus (a nuclear receptor) and exerts a variety of biological functions related to chronic pain and inflammation. Studies have shown that PEA interacts with distinct non-CB1/CB2 receptors, suggesting that PEA utilizes a unique “parallel” endocannabinoid signaling system. This concept was further supported by growing evidence that PEA production and inactivation can occur independently of AEA and 2-AG production and inactivation. Much of the biological effects of PEA on cells can be attributed to its affinity to PPAR (particularly PPAR-.alpha. and PPAR-.gamma.). PEA was shown to have an affinity to cannabinoid-like G-coupled receptors GPR55 and GPR119 as well as the transient receptor potential vanilloid type 1 receptor (TRPV1). PEA has been shown to have anti-inflammatory, anti-nociceptive, neuro-protective, and anti-convulsant properties.

### SUMMARY OF THE DISCLOSURE

In some embodiments, the present disclosure provides pharmaceutical compositions comprising LSD, or pharmaceutically acceptable salts thereof. In some embodiments, the present disclosure provides methods for preventing and/or treating a variety of conditions responsive to LSD treatment, such as alcoholism, depression, anxiety, headaches, and pain.

In some embodiments, the present disclosure provides pharmaceutical compositions comprising combinations of LSD, or pharmaceutically acceptable salts thereof, and N-acylethanolamines, or pharmaceutically acceptable salt thereof.

In some embodiments, these combinations comprise specific molar ratios between the respective active agents and/or by their dosages and may be employed in a variety of methods. In some embodiments, the present invention provides methods for preventing and/or treating a variety of conditions responsive to LSD treatment, such as alcoholism, depression, anxiety, headaches, and pain.

In certain embodiments, the molar ratio between the LSD and the N-acylethanolamine is between about 1:0.2 to about 1:5. In certain embodiments, the molar ratio between the LSD and the N-acylethanolamine is between about 1:0.5 to about 1:2. In certain embodiments, the molar ratio between the LSD and the N-acylethanolamine is between about 1:15 to about 1:1800. In certain embodiments, the molar ratio between the LSD and the N-acylethanolamine is between about 1:25 to about 1:450. In certain embodiments, the molar ratio between the LSD and the N-acylethanolamine is between about 1:50 to about 1:100. In certain embodiments, the molar ratio between the LSD and the N-acylethanolamine is about 1:50. In certain embodiments, the molar ratio between the LSD and the N-acylethanolamine is about 1:100. Each possibility represents a separate embodiment of the present disclosure.

In certain embodiments, the pharmaceutical composition comprises about 25-100 μg of LSD or a pharmaceutically acceptable salt thereof. Each possibility represents a separate embodiment of the present disclosure.

In certain embodiments, the pharmaceutical composition comprises about 200-1800 mg N-acylethanolamine or pharmaceutically acceptable salts thereof. In certain embodiments, the pharmaceutical composition comprises about 250 mg, about 500 mg, about 750 mg, about 1000 mg or about 1500 mg N-acylethanolamine or pharmaceutically acceptable salts thereof. Each possibility represents a separate embodiment of the present disclosure.

In certain embodiments, the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof. Each possibility represents a separate embodiment of the present disclosure. In certain embodiments, the N-acylethanolamine is PEA or a pharmaceutically acceptable salt thereof. In certain embodiments, the N-acylethanolamine consists of PEA or a pharmaceutically acceptable salt thereof. In certain embodiments, the N-acyl ethanol amine consists of PEA.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present disclosure. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

The present disclosure further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder or a spray. In certain embodiments, the dosage unit is formulated as a gel. In certain embodiments, the dosage unit is formulated as a powder. In certain embodiments, the dosage unit is formulated as a spray.

In other embodiments, the disclosure is directed to a method of treating alcoholism, comprising administrating to a subject in need thereof a therapeutically effective amount of a pharmaceutical composition comprising LSD, or a pharmaceutically acceptable salt thereof, thereby treating the condition.

In other embodiments, the disclosure is directed to a method of treating depression, comprising administrating to a subject in need thereof a therapeutically effective amount of a pharmaceutical composition comprising LSD, or a pharmaceutically acceptable salt thereof, thereby treating the condition.

In other embodiments, the disclosure is directed to a method of treating anxiety, comprising administrating to a subject in need thereof a therapeutically effective amount of a pharmaceutical composition comprising LSD, or a pharmaceutically acceptable salt thereof, thereby treating the condition.

In other embodiments, the disclosure is directed to a method of treating headaches, comprising administrating to a subject in need thereof a therapeutically effective amount of a pharmaceutical composition comprising LSD, or a pharmaceutically acceptable salt thereof, thereby treating the condition.

In other embodiments, the disclosure is directed to a method of treating pain, comprising administrating to a subject in need thereof a therapeutically effective amount of a pharmaceutical composition comprising LSD, or a pharmaceutically acceptable salt thereof, thereby treating the condition.

In certain embodiments, a therapeutically effective amount of a pharmaceutical composition comprising combinations of LSD, or a pharmaceutically acceptable salt thereof, and N-acylethanolamines, or a pharmaceutically acceptable salt thereof, is administered to a subject for treating alcoholism.

In certain embodiments, a therapeutically effective amount of a pharmaceutical composition comprising combinations of LSD, or a pharmaceutically acceptable salt thereof, and N-acylethanolamines, or a pharmaceutically acceptable salt thereof, is administered to a subject for treating depression.

In certain embodiments, a therapeutically effective amount of a pharmaceutical composition comprising combinations of LSD, or a pharmaceutically acceptable salt thereof, and N-acylethanolamines, or a pharmaceutically acceptable salt thereof, is administered to a subject for anxiety.

In certain embodiments, a therapeutically effective amount of a pharmaceutical composition comprising combinations of LSD, or a pharmaceutically acceptable salt thereof, and N-acylethanolamines, or a pharmaceutically acceptable salt thereof, is administered to a subject for treating headaches.

In certain embodiments, a therapeutically effective amount of a pharmaceutical composition comprising combinations of LSD, or a pharmaceutically acceptable salt thereof, and N-acylethanolamines, or a pharmaceutically acceptable salt thereof, is administered to a subject for treating pain.

In certain embodiments, the N-acylethanolamine increases the therapeutic potency of the LSD, or a pharmaceutically acceptable salt thereof, compared to the same pharmaceutical composition without the N-acylethanolamine. In certain embodiments, the N-acylethanolamine decreases the required therapeutic dosage of the LSD, or a pharmaceutically acceptable salt thereof, compared to the same pharmaceutical composition without the N-acylethanolamine. In certain embodiments, the N-acylethanolamine reduces at least one of the side-effects of the LSD, or a pharmaceutically acceptable salt thereof, compared to the same pharmaceutical composition without the N-acylethanolamine. In certain embodiments, the N-acylethanolamine expends the therapeutic window of the LSD, or a pharmaceutically acceptable salt thereof, compared to the same pharmaceutical composition without the N-acylethanolamine. In certain embodiments, the PEA or salt thereof increases the therapeutic potency of the THC or salt thereof compared to the same pharmaceutical composition without the PEA or salt thereof. In certain embodiments, the PEA (or pharmaceutically acceptable salts thereof) decreases the required therapeutic dosage of the THC (or pharmaceutically acceptable salts thereof) compared to the same pharmaceutical composition without the PEA (or pharmaceutically acceptable salts thereof). In certain embodiments, the PEA (or pharmaceutically acceptable salts thereof) reduces at least one of the side-effects of the THC (or pharmaceutically acceptable salts thereof) compared to the same pharmaceutical composition without the PEA (or pharmaceutically acceptable salts thereof). In certain embodiments, the PEA (or pharmaceutically acceptable salts thereof) expends the therapeutic window of the THC (or pharmaceutically acceptable salts thereof) compared to the same pharmaceutical composition without the PEA (or pharmaceutically acceptable salts thereof).

In certain embodiments of the methods described above, the LSD, or a pharmaceutically acceptable salt thereof, and the N-acylethanolamine, or a pharmaceutically acceptable salt thereof, are comprised in the same pharmaceutical composition. In certain embodiments of the methods described above, the LSD and the N-acylethanolamine or their pharmaceutically acceptable salts thereof are comprised in different pharmaceutical compositions.

In certain embodiments of the methods described above, the administration of the LSD and the N-acylethanolamine or their pharmaceutically acceptable salts thereof is repeated three times a day. In certain embodiments of the methods described above, the administration of the LSD and the N-acylethanolamine or their pharmaceutically acceptable salts is repeated twice a day. In certain embodiments of the methods described above, the administration of the LSD and the N-acylethanolamine or their pharmaceutically acceptable salts is repeated once a day. In certain embodiments of the methods described above, the administration of the LSD and the N-acylethanolamine or their pharmaceutically acceptable salts is repeated once every two days. In certain embodiments of the methods described above, the administration of the LSD and the N-acylethanolamine or their pharmaceutically acceptable salts is repeated once every three days.

In some embodiments, the present disclosure is directed to a pharmaceutical composition comprising LSD (or pharmaceutically acceptable salts thereof), and an N-acylethanolamine (or pharmaceutically acceptable salts thereof), and at least one pharmaceutically acceptable carrier and/or excipient.

In some embodiments, the pharmaceutical composition is a unit dosage from composition. In other embodiments, the pharmaceutical composition is a solid unit dosage form composition. In still other embodiments, the pharmaceutical composition is a liquid unit dosage form composition. In additional embodiments, the pharmaceutical composition is packaged as a single unit dose or as a plurality of single unit doses.

In further embodiments, the pharmaceutical composition further comprises at least on pharmaceutically acceptable carrier and/or excipient. In other embodiments, the pharmaceutical composition is a free-flowing powder, a tablet, a capsule, a lozenge, a liquid, a liquid concentrate, or a syrup.

In some embodiments, in the methods of the disclosure the pharmaceutical composition is a unit dosage form composition. In other embodiments, the amount of LSD or a pharmaceutically acceptable salt thereof in the unit dosage form ranges from about 25-100 μg.

In some embodiments, the pharmaceutical composition is administered orally. In some embodiments, the pharmaceutical composition is administered sublingually. In some embodiments, the pharmaceutical composition is administered intravenously.

Further embodiments and the full scope of applicability of the present disclosure will become apparent from the detailed description given hereinafter. However, the detailed description and specific examples, while indicating preferred embodiments of the disclosure, are given by way of illustration only, since various changes and modifications within the spirit and scope of the disclosure will become apparent to those skilled in the art from this detailed description.

### DETAILED DESCRIPTION OF THE DISCLOSURE

### Definitions

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbol “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

The present disclosure provides, in one aspect, a pharmaceutical composition comprising a therapeutically-effective amount of a mixture of LSD (or pharmaceutically acceptable salts thereof) and at least one N-acylethanolamine (or pharmaceutically acceptable salts thereof).

The present disclosure provides, in another aspect, a pharmaceutical composition comprising a therapeutically-effective amount of a mixture of LSD (or pharmaceutically acceptable salts thereof) and at least one N-acylethanolamine (or pharmaceutically acceptable salts thereof), wherein the molar ratio between the LSD and the N-acylethanolamine (or pharmaceutically acceptable salts thereof) is between about 1:0.2 to about 1:2000.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, oils such as vegetable oils or fish oils, and polyethylene glycols.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “N-acylethanolamine” as used herein generally refers to a type of fatty acid amide, lipid-derived signaling molecules, formed when one of several types of acyl group is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines. The suffixes -amine and -amide in these names each refer to the single nitrogen atom of ethanolamine that links the compound together: it is termed “amine” in ethanolamine because it is considered as a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “derivative” as used herein means a compound whose core structure is the same as, or closely resembles that of an N-acylethanolamine compound, but which has a chemical or physical modification, such as different or additional side groups.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction.

In certain embodiments, the molar ratio between the LSD and the N-acylethanolamine (or pharmaceutically acceptable salts thereof) is between about 1:0.2 to about 1:5. In certain embodiments, the molar ratio between the LSD and the N-acylethanolamine (or pharmaceutically acceptable salts thereof) is between about 1:0.22 to about 1:4.5, about 1:0.25 to about 1:4, between about 1:0.28 to about 1:3.5, between about 1:0.33 to about 1:3, between about 1:0.4 to about 1:2.5, between about 1:0.5 to about 1:2 or about 1:1. Each possibility represents a separate embodiment of the present disclosure.

In certain embodiments, the molar ratio between the LSD and the N-acylethanolamine (or pharmaceutically acceptable salts thereof) is between about 1:15 to about 1:1800. In certain embodiments, the molar ratio between the LSD and the N-acylethanolamine is between about 1:16 to about 1:1700, about 1:17 to about 1:1600, about 1:18 to about 1:1500, about 1:19 to about 1:1400, about 1:20 to about 1:1300, about 1:21 to about 1:1200, about 1:22 to about 1:1100, about 1:23 to about 1:1000, about 1:24 to about 1:900, about 1:15 to about 1:800, about 1:16 to about 1:700, about 1:17 to about 1:600, about 1:18 to about 1:500, about 1:19 to about 1:490, about 1:20 to about 1:480, about 1:21 to about 1:470, or about 1:22 to about 1:460. Each possibility represents a separate embodiment of the present disclosure. In certain embodiments, the molar ratio between the LSD and the N-acylethanolamine is between about 1:25 to about 1:450. In certain embodiments, the molar ratio between the LSD and the N-acylethanolamine is between about 1:10 to about 1:500, about 1:15 to about 1:450, about 1:20 to about 1:400, about 1:25 to about 1:350, about 1:30 to about 1:300, about 1:35 to about 1:250, about 1:40 to about 1:200, or about 1:45 to about 1:150. Each possibility represents a separate embodiment of the present disclosure. In certain embodiments, the molar ratio between the LSD and the N-acylethanolamine is between about 1:50 to about 1:100. In certain embodiments, the molar ratio between the LSD and the N-acylethanolamine is about 1:10. In certain embodiments, the molar ratio between the LSD and the N-acylethanolamine is about 1:20. In certain embodiments, the molar ratio between the LSD and the N-acylethanolamine is about 1:30. In certain embodiments, the molar ratio between the LSD and the N-acylethanolamine is about 1:40. In certain embodiments, the molar ratio between the LSD and the N-acylethanolamine is about 1:50. In certain embodiments, the molar ratio between the LSD and the N-acylethanolamine is about 1:60. In certain embodiments, the molar ratio between the LSD and the N-acylethanolamine is about 1:70. In certain embodiments, the molar ratio between the LSD and the N-acylethanolamine is about 1:80. In certain embodiments, the molar ratio between the LSD and the N-acylethanolamine is about 1:90. In certain embodiments, the molar ratio between the LSD and the N-acylethanolamine is about 1:100. In certain embodiments, the molar ratio between the LSD and the N-acylethanolamine is about 1:110. In certain embodiments, the molar ratio between the LSD and the N-acylethanolamine is about 1:120. In certain embodiments, the molar ratio between the LSD and the N-acylethanolamine is about 1:130. In certain embodiments, the molar ratio between the LSD and the N-acylethanolamine is about 1:140. In certain embodiments, the molar ratio between the LSD and the N-acylethanolamine is about 1:150. In certain embodiments, the molar ratio between the LSD and the N-acylethanolamine is about 1:160. In certain embodiments, the molar ratio between the LSD and the N-acylethanolamine is about 1:170. In certain embodiments, the molar ratio between the LSD and the N-acylethanolamine is about 1:180. In certain embodiments, the molar ratio between the LSD and the N-acylethanolamine is about 1:190. In certain embodiments, the molar ratio between the LSD and the N-acylethanolamine is about 1:200. In certain embodiments, the molar ratio between the LSD and the N-acylethanolamine is at least about 1:10, at least about 1:20, at least about 1:30, at least about 1:40, at least about 1:50, at least about 1:60, at least about 1:70, at least about 1:80, at least about 1:90, or at least about 1:100. Each possibility represents a separate embodiment of the present disclosure.

In certain embodiments, the pharmaceutical composition comprises about 0.5-10 mg LSD or a salt thereof. In certain embodiments, the pharmaceutical composition comprises about 1-9.5 mg, about 1.5-9 mg, about 2-8.5 mg, about 2.5-8 mg, about 3-7.5 mg, about 3.5-7 mg, about 4-6.5 mg, about 4.5-6 mg or about 5-5.5 mg LSD or a salt thereof. In certain embodiments, the pharmaceutical composition comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg LSD or a salt thereof. Each possibility represents a separate embodiment of the present disclosure. In certain embodiments, the pharmaceutical composition comprises less than about 0.5 mg, less than about 1 mg, less than about 1.5 mg, less than about 2 mg, less than about 2.5 mg, less than about 3 mg, less than about 3.5 mg, less than about 4 mg, less than about 4.5 mg, less than about 5 mg, less than about 5.5 mg, less than about 6 mg, less than about 6.5 mg, less than about 7 mg, less than about 7.5 mg, less than about 8 mg, less than about 8.5 mg, less than about 9 mg, less than about 9.5 mg or about 10 mg LSD or a pharmaceutically acceptable salt thereof. Each possibility represents a separate embodiment of the present disclosure. In certain embodiments, the pharmaceutical composition comprises about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 0.5 mg to about 5.5 mg, about 0.5 mg to about 6 mg, about 0.5 mg to about 6.5 mg, about 0.5 mg to about 7 mg, about 0.5 mg to about 7.5 mg, about 0.5 mg to about 8 mg, about 0.5 mg to about 8.5 mg, about 0.5 mg to about 9 mg or about 0.5 mg to about 9.5 mg LSD or a pharmaceutically acceptable salt thereof. Each possibility represents a separate embodiment of the present disclosure.

In certain embodiments, the pharmaceutical composition comprises about 200-1800 mg N-acylethanolamine or a pharmaceutically acceptable salt thereof. In certain embodiments, the pharmaceutical composition comprises about 250-1550 mg, about 300-1200 mg, about 350-950 mg, about 400-700 mg, about 450-600 mg or about 500-550 mg N-acylethanolamine or a pharmaceutically acceptable salt thereof. Each possibility represents a separate embodiment of the present disclosure. In certain embodiments, the pharmaceutical composition comprises at least about 50 mg, at least about 100 mg, at least about 150 mg, at least about 200 mg, at least about 250 mg, at least about 300 mg, at least about 350 mg, at least about 400, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg or at least about 1800 mg N-acylethanolamine or a pharmaceutically acceptable salt thereof. In certain embodiments, the pharmaceutical composition comprises about 50 mg, about 100 mg, about 150 mg, about 200 mg, about 250 mg, about 300 mg, about 350 mg, about 400, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg or about 1800 mg N-acylethanolamine or a pharmaceutically acceptable salt thereof. Each possibility represents a separate embodiment of the present disclosure.

In certain embodiments, the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), pharmaceutically acceptable salts thereof and any combination thereof. Each possibility represents a separate embodiment of the present disclosure. In certain embodiments, the N-acylethanolamine is PEA or a pharmaceutically acceptable salt thereof. In certain embodiments, the N-acylethanolamine consists of PEA or a pharmaceutically acceptable salt thereof. In certain embodiments, the N-acylethanolamine consists of PEA.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present disclosure. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

Techniques for formulation and administration of drugs are well known in the art, and may be found, e.g. in “Remington's Pharmaceutical Sciences,” Mack Publishing Co., Easton, Pa. Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal and sublingual administration, the compositions may take the form of tablets or lozenges formulated in conventional manner or in adhesive carriers. Alternatively, the active ingredient may be in powder form for constitution with a suitable vehicle, e.g., a sterile, pyrogen-free, water-based solution, before use.

Pharmaceutical compositions suitable for use in the context of the present disclosure include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein. More specifically, a “therapeutically effective amount of a mixture” means an amount of at least two active ingredients, wherein each one of the active ingredients independently may not be in a therapeutically effective amount or wherein both of the active ingredients may not be in a therapeutically effective amount, the mixture is nevertheless effective to prevent, alleviate, or ameliorate symptoms or side effects of a disease or disorder, or prolong the survival of the subject being treated. The term “mixture” as used herein refers to a non-covalent combination of two molecules.

For any preparation used in the methods of the disclosure, the dosage or the therapeutically effective amount can be estimated initially from in vitro, in vivo and cell culture assays. For example, a dose can be formulated in animal models to achieve a desired concentration or titer. Such information can be used to more accurately determine useful doses in humans. The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Continuous daily dosing may not be required; a therapeutic regimen may require cycles, during which time a drug is not administered, or therapy may be provided on an as-needed basis during periods of acute disease worsening. Dosage escalation may or may not be required; a therapeutic regimen may require reduction in medication dosage. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1). Depending on the severity and responsiveness of the condition to be treated, dosing can be of a single or a plurality of administrations, with course of treatment lasting from several days to several weeks, or until cure is effected or diminution of the disease state is achieved.

The present disclosure further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder or a spray. In certain embodiments, the dosage unit is formulated as a gel. In certain embodiments, the dosage unit is formulated as a powder. In certain embodiments, the dosage unit is formulated as a spray.

The present disclosure further provides, in another aspect, a pharmaceutical composition or a dosage unit as described above for use in a method for preventing or treating a condition amenable to prevention or treatment by LSD or a pharmaceutically acceptable salt thereof.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the disclosure.

The term “acute” refers to a condition with a relatively short, severe course.

The term “chronic” as used herein means that the length of time of the diseases or conditions of the disclosure can be weeks, months, or possibly years. The intensity of the diseases or conditions can differentiate according to various conditions such as patient age, temperature, season, type of disease, etc.

The term “about” as used herein in relation to a value, a plurality of values or a range of values defined by a lowest and highest values means a value which is 10% lower and/or higher than the corresponding value, plurality of values or range of values. For example, the phrase “about 1” means “0.9 to 1.1”, the phrase “about 1 or 2” means “0.9 to 1.1 or 1.8 to 2.2”, and the phrase “about 1 to about 2” means “0.9 to 2.2”.

The terms “comprises”, “comprising”, “includes”, “including”, “having” and their conjugates mean “including but not limited to.”

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and the following Table for Equivalent Surface Area Dosage Factors).

TABLE 1
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the present disclosure provides pharmaceutical compositions comprising LSD, or pharmaceutically acceptable salts thereof. In some embodiments, the present disclosure provides methods for preventing and/or treating a variety of conditions responsive to LSD treatment, such as alcoholism, depression, anxiety, headaches, and pain.

In some embodiments, the present disclosure provides pharmaceutical compositions comprising combinations of LSD, or pharmaceutically acceptable salts thereof, and N-acylethanolamines, or pharmaceutically acceptable salt thereof.

In some embodiments, these combinations comprise specific molar ratios between the respective active agents and/or by their dosages and may be employed in a variety of methods.

In some embodiments, the unit dosage form comprises from 25-100 μg of lysergic acid diethylamide or a pharmaceutically acceptable salt thereof.

In some embodiments, the present invention provides methods for preventing and/or treating a variety of conditions responsive to LSD treatment, such as alcoholism, depression, anxiety, headaches, and pain.

One or more embodiments of the present disclosure include the following embodiments 1 to 39:
1. A pharmaceutical composition comprising lysergic acid diethylamide, or a pharmaceutically acceptable salt thereof, and at least one pharmaceutically acceptable carrier and/or excipient.
2. The pharmaceutical composition of embodiment 1, wherein the composition is a unit dosage from composition.
3. The pharmaceutical composition of embodiment 2, wherein the pharmaceutical composition is a solid unit dosage form composition.
4. The pharmaceutical composition of embodiment 2, wherein the pharmaceutical composition is a liquid unit dosage form composition.
5. The pharmaceutical composition of embodiment 2, wherein the pharmaceutical composition is packaged as a single unit dose or as a plurality of single unit doses.
6. The pharmaceutical composition of embodiment 2, wherein the unit dosage form comprises from 25-100 μg of lysergic acid diethylamide or a pharmaceutically acceptable salt thereof.
7. The pharmaceutical composition of embodiment 1, wherein the pharmaceutical composition is formulated for oral administration, sublingual administration, or intravenous administration.
8. A method of treating alcoholism, comprising administrating to a subject in need thereof a therapeutically effective amount of a pharmaceutical composition comprising lysergic acid diethylamide, or a pharmaceutically acceptable salt thereof.
9. A method of treating depression, comprising administrating to a subject in need thereof a therapeutically effective amount of a pharmaceutical composition comprising lysergic acid diethylamide, or a pharmaceutically acceptable salt thereof.
10. A method of treating anxiety, comprising administrating to a subject in need thereof a therapeutically effective amount of a pharmaceutical composition comprising lysergic acid diethylamide, or a pharmaceutically acceptable salt thereof.
11. A method of treating headaches, comprising administrating to a subject in need thereof a therapeutically effective amount of a pharmaceutical composition comprising lysergic acid diethylamide, or a pharmaceutically acceptable salt thereof.
12. A method of treating pain, comprising administrating to a subject in need thereof a therapeutically effective amount of a pharmaceutical composition comprising lysergic acid diethylamide, or a pharmaceutically acceptable salt thereof.
13. The method of any of embodiments 8, 9, 10, 11, or 12, wherein the pharmaceutical composition further comprises at least on pharmaceutically acceptable carrier and/or excipient.
14. The method of any of embodiments 8, 9, 10, 11, or 12, wherein the pharmaceutical composition is a free-flowing powder, a tablet, a capsule, a lozenge, a liquid, a liquid concentrate, or a syrup.
15. The method of any of embodiments 8, 9, 10, 11, or 12, wherein the pharmaceutical composition is a unit dosage form composition.
16. The method of any of embodiments 8, 9, 10, 11, or 12, wherein an amount of lysergic acid diethylamide or a pharmaceutically acceptable salt thereof in the unit dosage form ranges from about 25-100 μg.
17. The method of any of embodiments 8, 9, 10, 11, or 12, wherein the pharmaceutical composition is administered orally or intravenously.
18. A pharmaceutical composition comprising lysergic acid diethylamide or a pharmaceutically acceptable salt thereof, and an N-acylethanolamine or a pharmaceutically acceptable salt thereof, and at least one pharmaceutically acceptable carrier and/or excipient.
19. The pharmaceutical composition of embodiment 18, wherein the composition is a unit dosage from composition.
20. The pharmaceutical composition of embodiment 19, wherein the pharmaceutical composition is a solid unit dosage form composition.
21. The pharmaceutical composition of embodiment 19, wherein the pharmaceutical composition is a liquid unit dosage form composition.
22. The pharmaceutical composition of embodiment 19, wherein the pharmaceutical composition is packaged as a single unit dose or as a plurality of single unit doses.
23. The pharmaceutical composition of embodiment 19, wherein the unit dosage form comprises from 25-100 μg of lysergic acid diethylamide or a pharmaceutically acceptable salt thereof.
24. The pharmaceutical composition of embodiment 19, wherein the unit dosage form comprises from 50 mg to 1800 mg of N-acylethanolamine or a pharmaceutically acceptable salt thereof.
25. The pharmaceutical composition of embodiment 18, wherein the pharmaceutical composition is formulated for oral administration or intravenous administration.
26. A method of treating alcoholism, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.
27. A method of treating depression, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.
28. A method of treating anxiety, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.
29. A method of treating headaches, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.
30. A method of treating pain, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.
31. The method of any of embodiments 26, 27, 28, 29, or 30, wherein said the pharmaceutical composition further comprises at least on pharmaceutically acceptable carrier and/or excipient.
32. The method of any of embodiments 26, 27, 28, 29, or 30, wherein the pharmaceutical composition is a free-flowing powder, a tablet, a capsule, a lozenge, a liquid, a liquid concentrate, or a syrup.
33. The method of any of embodiments 26, 27, 28, 29, or 30, wherein the pharmaceutical composition is a unit dosage form composition.
34. The method of any of embodiments 26, 27, 28, 29, or 30, wherein an amount of lysergic acid diethylamide or a pharmaceutically acceptable salt thereof in the unit dosage form ranges from 25-100 μg.
35. The method of any of embodiments 26, 27, 28, 29, or 30, wherein the pharmaceutical composition is administered orally or intravenously.
36. The pharmaceutical composition of any preceding embodiments, wherein the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof.
37. The method of any preceding embodiments, wherein the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof.
38. The pharmaceutical composition of any preceding embodiments, wherein the N-acylethanolamine is PEA or a pharmaceutically acceptable salt thereof.
39. The method of any preceding embodiments, wherein the N-acylethanolamine is PEA or a pharmaceutically acceptable salt thereof.

What is claimed is:

1. A pharmaceutical composition comprising lysergic acid diethylamide, or a pharmaceutically acceptable salt thereof, and at least one pharmaceutically acceptable carrier and/or excipient.

2. The pharmaceutical composition of claim 1, wherein the composition is a unit dosage from composition.

3. The pharmaceutical composition of claim 2, wherein the pharmaceutical composition is a solid unit dosage form composition.

4. The pharmaceutical composition of claim 2, wherein the pharmaceutical composition is a liquid unit dosage form composition.

5. The pharmaceutical composition of claim 2, wherein the pharmaceutical composition is packaged as a single unit dose or as a plurality of single unit doses.

6. The pharmaceutical composition of claim 2, wherein the unit dosage form comprises from 25-100 μg of lysergic acid diethylamide or a pharmaceutically acceptable salt thereof.

7. The pharmaceutical composition of claim 1, wherein the pharmaceutical composition is formulated for oral administration, sublingual administration, or intravenous administration.

8. A method of treating alcoholism, comprising administrating to a subject in need thereof a therapeutically effective amount of a pharmaceutical composition comprising lysergic acid diethylamide, or a pharmaceutically acceptable salt thereof.

9. A method of treating depression, comprising administrating to a subject in need thereof a therapeutically effective amount of a pharmaceutical composition comprising lysergic acid diethylamide, or a pharmaceutically acceptable salt thereof.

10. A method of treating anxiety, comprising administrating to a subject in need thereof a therapeutically effective amount of a pharmaceutical composition comprising lysergic acid diethylamide, or a pharmaceutically acceptable salt thereof.

11. A method of treating headaches, comprising administrating to a subject in need thereof a therapeutically effective amount of a pharmaceutical composition comprising lysergic acid diethylamide, or a pharmaceutically acceptable salt thereof.

12. A method of treating pain, comprising administrating to a subject in need thereof a therapeutically effective amount of a pharmaceutical composition comprising lysergic acid diethylamide, or a pharmaceutically acceptable salt thereof.

13. The method of claim 8, 9, 10, 11, or 12, wherein the pharmaceutical composition further comprises at least on pharmaceutically acceptable carrier and/or excipient.

14. The method of claim 8, 9, 10, 11, or 12, wherein the pharmaceutical composition is a free-flowing powder, a tablet, a capsule, a lozenge, a liquid, a liquid concentrate, or a syrup.

15. The method of claim 8, 9, 10, 11, or 12, wherein the pharmaceutical composition is a unit dosage form composition.

16. The method of claim 8, 9, 10, 11, or 12, wherein an amount of lysergic acid diethylamide or a pharmaceutically acceptable salt thereof in the unit dosage form ranges from about 25-100 μg.

17. The method of claim 8, 9, 10, 11, or 12, wherein the pharmaceutical composition is administered orally or intravenously.

18. A pharmaceutical composition comprising lysergic acid diethylamide or a pharmaceutically acceptable salt thereof, and an N-acylethanolamine or a pharmaceutically acceptable salt thereof, and at least one pharmaceutically acceptable carrier and/or excipient.

19. The pharmaceutical composition of claim 18, wherein the composition is a unit dosage from composition.

20. The pharmaceutical composition of claim 19, wherein the pharmaceutical composition is a solid unit dosage form composition.

21. The pharmaceutical composition of claim 19, wherein the pharmaceutical composition is a liquid unit dosage form composition.

22. The pharmaceutical composition of claim 19, wherein the pharmaceutical composition is packaged as a single unit dose or as a plurality of single unit doses.

23. The pharmaceutical composition of claim 19, wherein the unit dosage form comprises from 25-100 μg of lysergic acid diethylamide or a pharmaceutically acceptable salt thereof.

24. The pharmaceutical composition of claim 19, wherein the unit dosage form comprises from 50 mg to 1800 mg of N-acylethanolamine or a pharmaceutically acceptable salt thereof.

25. The pharmaceutical composition of claim 18, wherein the pharmaceutical composition is formulated for oral administration or intravenous administration.

26. A method of treating alcoholism, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.

27. A method of treating depression, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.

28. A method of treating anxiety, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.

29. A method of treating headaches, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.

30. A method of treating pain, comprising administrating to a subject in need thereof a therapeutically effective amount of a pharmaceutical composition comprising lysergic acid diethylamide, or a pharmaceutically acceptable salt thereof, and an N-acylethanolamine, or a pharmaceutically acceptable salt thereof.

31. The method of claim 26, 27, 28, 29, or 30, wherein said the pharmaceutical composition further comprises at least on pharmaceutically acceptable carrier and/or excipient.

32. The method of claim 26, 27, 28, 29, or 30, wherein the pharmaceutical composition is a free-flowing powder, a tablet, a capsule, a lozenge, a liquid, a liquid concentrate, or a syrup.

33. The method of claim 26, 27, 28, 29, or 30, wherein the pharmaceutical composition is a unit dosage form composition.

34. The method of claim 26, 27, 28, 29, or 30, wherein an amount of lysergic acid diethylamide or a pharmaceutically acceptable salt thereof in the unit dosage form ranges from 25-100 μg.

35. The method of claim 26, 27, 28, 29, or 30, wherein the pharmaceutical composition is administered orally or intravenously.

36. The pharmaceutical composition of any preceding claim, wherein the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof.

37. The method of any preceding claim, wherein the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof.

38. The pharmaceutical composition of any preceding claim, wherein the N-acylethanolamine is PEA or a pharmaceutically acceptable salt thereof.

39. The method of any preceding claim, wherein the N-acylethanolamine is PEA or a pharmaceutically acceptable salt thereof.
